(19) United States
(12) Patent Application Publication
(10) Pub. No.: US 2003/0191096 A1
(43) Pub. Date: Oct. 9, 2003

(54) Method of hormonal therapy

(76) Inventors: Thomas W. Leonard, Wilmington, NC (US); R. Forrest Waldon, Wilmington, NC (US)

Correspondence Address: MYERS BIGEL SIBLEY & SAJOVEC, PO BOX 37428, RALEIGH, NC, 27627, US

(21) Appl. No.: 10/268,008

(22) Filed: Oct. 9, 2002

Related U.S. Application Data
(60) Provisional application No. 60/369,635, filed on Apr. 3, 2002.

(51) Int. Cl. A61K 31/56

(52) U.S. Cl. 514/170; 514/171

(57) ABSTRACT

The present invention combines the administration of estrogens with the administration of non-aromatizing androgens to treat frailty in women undergoing estrogen replacement therapy.

### CROSS-REFERENCE TO RELATED APPLICATIONS

This application claims priority to U.S. Provisional Application Serial No. 60/369,635 which was filed in the United States Patent and Trademark Office on Apr. 3, 2002, the disclosure of which is incorporated herein by reference in its entirety.

### FIELD OF THE INVENTION

The present invention generally relates to a method of treating sexual dysfunction in women, particularly in menopausal and post menopausal women.

### BACKGROUND OF THE INVENTION

Menopause, which typically occurs in women during middle age, is often described as an ovarian shutdown. It is often associated with a profound decrease in circulating levels of estrogens. A decrease in androgen levels occurs on a much slower basis approximately five years after the precipitous decline in estrogen levels. An exception to this characteristic occurs when a woman undergoes surgical menopause caused by the removal of both ovaries. In these cases a precipitous drop occurs in both estrogen and androgen levels. There are a large variety of disorders and conditions that are attributed to these reductions of hormone levels. Exemplary disorders and conditions include sexual dysfunctions such as dryness and atrophy of the vagina, dyspareunia, loss of desire, anaorgasmia and non-sexual dysfunction disorders including parathesia, hot flashes, osteoporosis, and an increase in cardiovascular disease. Administration of estrogens and/or androgens, so-called “hormone replacement therapy”, to postmenopausal women continues to be the primary treatment of such disorders and conditions associated with menopause. Testosterone and methyltestosterone are androgens that are used in these treatments. These androgens have well known problems and side effects as therapies. Testosterone is not absorbed orally. Methyltestosterone is associated with liver toxicity. Both of these compounds promote development of secondary male sex characteristics at therapeutic doses, including body hair growth, deepening of the voice and enlargement of the clitoris. The present applicants have previously made known that non-aromatizing androgens provide less development of secondary sex characteristics. Thus, it may be useful to use these agents in the treatment of female sexual dysfunction.

As women age, problems associated with the decline in hormone production may increase. A decline in hormone production may cause a decrease in bone mineral density which results in osteoporosis and other bone disorders as well as increasing in susceptibility to fractures. Thus, women may see an increase in frailty or weakness and may experience geriatric wasting as they age. For some women, wellness issues associated with hormonal deficiencies may arise, such as poor quality of sleep, lack of energy and difficulties with balance. The decline in hormone production may further result in female sexual dysfunction, including problems associated with a decline in libido and desire. Thus, it would be desirable to provide to women with treatment that would address the symptoms and conditions created by a hormone deficiency as well as reducing any potential side effects from such therapy.

### SUMMARY OF THE INVENTION

The present invention addresses the administration of non-aromatizing androgens to allow chronic therapy in postmenopausal women. It was determined that non-aromatizing androgens have excellent androgen effects in addition to the well established anabolic effects and may be used to treat women with an androgen deficiency syndrome. Yet, the non-aromatizing androgens do not exhibit the level of hirsutism found with therapy utilizing aromatizing androgens. Therefore, the present invention illustrates that an androgen replacement therapy is best carried out with non-aromatizing androgens even in patients with and without intact uteri.

In one embodiment, the method of treating female sexual dysfunction includes administering, continuously and uninterruptedly, a therapeutically effective amount of non-aromatizing androgen in daily dosages. In another embodiment the method of treating frailty includes administering, continuously and uninterruptedly, a therapeutically effective amount of non-aromatizing androgen in daily dosages. In another embodiment the method of treating female sexual dysfunction and frailty includes administering, continuously and uninterruptedly, a therapeutically effective amount of estrogen and non-aromatizing androgen in daily dosages. In another embodiment, the method of treating sexual dysfunction and frailty includes cyclically administering the non-aromatizing androgen compound and continuously and uninterruptedly administering the estrogenic compound. Another embodiment utilizes administering a non-aromatizing androgen combined with an aromatizing androgen.

### DETAILED DESCRIPTION OF THE PREFERRED EMBODIMENTS

The invention will now be described with reference to the embodiments set forth herein. These embodiments are intended to illustrate the invention and are not meant to limit the scope of the invention.

In one aspect, the invention relates to a pharmaceutical composition. The pharmaceutical composition comprises a therapeutically effective amount of a non-aromatizing androgenic compound, and a pharmaceutically acceptable carrier. Additionally, the compound may contain an estrogenic and/or a progestational agent.

A “therapeutically effective” amount as used herein is an amount of an estrogenic compound and a non-aromatizing androgenic compound that is sufficient to treat hormonal deficiencies in a subject. The therapeutically effective amount will vary with the age and physical condition of the patient, the severity of the treatment, the duration of the treatment, the nature of any concurrent treatment, the pharmaceutically acceptable carrier used and like factors within the knowledge and expertise of those skilled in the art. Pharmaceutically acceptable carriers are preferably solid dosage forms such as tablets or capsules, liquids, transdermal patches and other acceptable carriers, the selection of which are known in the art.

Liquid preparations for oral administration may be prepared in the form of syrups or suspensions, e.g., solutions containing an active ingredient, sugar, and a mixture of ethanol, water, glycerol, and propylene glycol. If desired, such liquid preparations may contain coloring agents, flavoring agents, and saccharin. Thickening agents such as carboxymethylcellulose may also be used.

Suitable non-aromatizing androgenic compounds include oxandrolone, oxymetholone, stanozolol, stanozolone, danazol, and combinations of any of the foregoing. Preferably, the therapeutically effective amount of the non-aromatizing androgenic compound is about 0.1 to about 10 mg based on oral dose equivalents of oxandrolone. For women suffering from androgen deficiency the oral dosage equivalents of oxandrolone is about 0.5 to 4 mg of oxandrolone per day. Additionally, subjects may be given a combination of an androgenic compound that is non-aromatizing as combined with an androgenic compound that may aromatize. Preferably the amount of the non-aromatizing compound is at least 50% of the oral dosage given to a subject.

Estrogen levels are related to the general physiological health of postmenopausal women and may be very helpful additives in the treatment of sexual dysfunction. Also they contribute to health of the vagina, provide local vasodilation effects and stimulate mucous production. Additionally, they exert positive CNS effects on hot flashes, and improve nerve transmission which is believed to delay various types of dementia. They have positive cardiovascular effects by improving lipid levels and promoting vasodilation and relaxation. Suitable estrogenic compounds include estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, and the estrogen ketones and their corresponding 17α- and 17-β hydroxy derivatives. The estrogenic compounds may also be present as conjugated estrogens. The conjugates may be various conjugates understood by those skilled in the art, including, but not limited to, sulfate and glucuronide. The most preferred estrogen conjugates are estrogen sulfates. Approximately 1.0 mg of 17β estradiol is equivalent to 0.625 mg of conjugated estrogens. The estrogenic compounds can be derived from natural and synthetic sources. Preferably, the therapeutically effective amount of estrogenic compound is about 0.05 to about 3 mg, and preferably about 0.5 to about 2 mg based on oral dose equivalents of estradiol.

The androgen formulations can be, for example, in the form of tablets; effervescent tablets; pills; powders; elixirs; suspensions; emulsions; solutions; syrups; soft and hard gelatin capsules; transdermal patches; topical gels, creams and the like; vaginal suppositories; sterile injectable solutions; and sterile packaged powders, sublingual tablets, buccal tablets and buccal adhesive systems.

The estrogen formulations can be, for example, in the form of tablets; effervescent tablets; pills; powders; elixirs; suspensions; emulsions; solutions; syrups; soft and hard gelatin capsules; transdermal patches; topical gels, creams and the like; vaginal suppositories; sterile injectable solutions; and sterile packaged powders, sublingual tablets, buccal tablets and buccal adhesive systems.

Additionally, as previously stated, a progestational agent may be used in combination with the estrogenic compound. Examples of progestational agents are set forth in U.S. Pat. No. Re. 36,247 to Plunkett et al. Examples include, but are not limited to, laevo-norgestrel, dl-norgestrel, norethindrone (norethisterone), norethindrone (norethisterone) acetate, megestrol acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, and cyproterone acetate.

In certain embodiments, the drug product is present in a solid pharmaceutical composition that may be suitable for oral administration. A solid composition of matter according to the present invention may be formed and may be mixed with and/or diluted by an excipient. The solid composition of matter may also be enclosed within a carrier which may be, for example, in the form of a capsule, sachet, tablet, paper, or other container. When the excipient serves as a diluent, it may be a solid, semi-solid, or liquid material which acts as a vehicle, carrier, or medium for the composition of matter.

Various suitable excipients will be understood by those skilled in the art and may be found in the National Formulary, 19: 2404-2406 (2000), the disclosure of pages 2404 to 2406 being incorporated herein in their entirety. Examples of suitable excipients include, but are not limited to, starches, gum arabic, calcium silicate, microcrystalline cellulose, methacrylates, shellac, polyvinylpyrrolidone, cellulose, water, syrup, and methylcellulose. The drug product formulations can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to, mannitol, sorbitol, xylitol, sucrose, maltose, glucose, lactose, dextrose, and the like. Suitable buffers encompass, but are not limited to, phosphate, citrate, tartrate, succinate, and the like. Other inert fillers which may be used encompass those which are known in the art and are useful in the manufacture of various dosage forms. If desired, the solid formulations may include other components such as bulking agents and/or granulating agents, and the like. The drug products of the invention may be formulated so as to provide quick, sustained, or delayed release of the active ingredient after administration to the patient by employing procedures well known in the art.

To form tablets for oral administration, the composition of matter of the present invention may be made by a direct compression process. In this process, the active drug ingredients may be mixed with a solid, pulverant carrier such as, for example, lactose, saccharose, sorbitol, mannitol, starch, amylopectin, cellulose derivatives or gelatin, and mixtures thereof, as well as with an antifriction agent such as, for example, magnesium stearate, calcium stearate, and polyethylene glycol waxes. The mixture may then be pressed into tablets using a machine with the appropriate punches and dies to obtain the desired tablet size. The operating parameters of the machine may be selected by the skilled artisan. Alternatively, tablets for oral administration may be formed by a wet granulation process. Active drug ingredients may be mixed with excipients and/or diluents. The solid substances may be ground or sieved to a desired particle size. A binding agent may be added to the drug. The binding agent may be suspended and homogenized in a suitable solvent. The active ingredient and auxiliary agents may also be mixed with the binding agent solution. The resulting dry mixture is moistened with the solution uniformly. The moistening typically causes the particles to aggregate slightly, and the resulting mass is pressed through a stainless steel sieve having a desired size. The mixture is then dried in controlled drying units for the determined length of time necessary to achieve a desired particle size and consistency. The granules of the dried mixture are sieved to remove any powder. To this mixture, disintegrating, antifriction, and/or anti-adhesive agents are added. Finally, the mixture is pressed into tablets using a machine with the appropriate punches and dies to obtain the desired tablet size. The operating parameters of the machine may be selected by the skilled artisan.

If coated tablets are desired, the above prepared core may be coated with a concentrated solution of sugar or cellulosic polymers, which may contain gum arabic, gelatin, talc, titanium dioxide, or with a lacquer dissolved in a volatile organic solvent or a mixture of solvents. To this coating various dyes may be added in order to distinguish among tablets with different active compounds or with different amounts of the active compound present. In a particular embodiment, the active ingredient may be present in a core surrounded by one or more layers including enteric coating layers.

Soft gelatin capsules may be prepared in which capsules contain a mixture of the active ingredient and vegetable oil. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, and/or gelatin.

In one preferred embodiment, the formulation is in the form of orally-administered tablets which contain the composition of matter of the present invention as set forth herein along with the following inactive ingredients: calcium phosphate tribasic, calcium sulfate, carnauba wax, cellulose, glyceryl monooleate, lactose, magnesium stearate, methylcellulose, pharmaceutical glaze, polyethylene glycol, stearic acid, sucrose, and titanium dioxide. Such ingredients may be present in amounts similar to those present in Premarin® (conjugated estrogens tablets, USP) made commercially available by Wyeth-Ayerst Laboratories of Philadelphia, Pa. Tablets employing the active ingredients of the invention may contain excipients similar to those contained in the 0.3 mg, 0.625 mg, and 1.25 mg tablets of Premarin® (conjugated estrogens tablets, USP).

Liquid preparations for oral administration may be prepared in the form of syrups or suspensions, e.g., solutions containing an active ingredient, sugar, and a mixture of ethanol, water, glycerol, and propylene glycol. If desired, such liquid preparations may contain coloring agents, flavoring agents, and saccharin. Thickening agents such as carboxymethylcellulose may also be used.

In the event that the above formulations are to be used for parenteral administration, such a formulation may comprise sterile aqueous injection solutions, non-aqueous injection solutions, or both comprising the composition of matter of the present invention. When aqueous injection solutions are prepared, the composition of matter may be present as a water soluble pharmaceutically acceptable salt. Parenteral preparations may contain anti-oxidants, buffers, bacteriostats, and solutes which render the formulation isotonic with the blood of the intended recipient. Aqueous and non-aqueous sterile suspensions may include suspending agents and thickening agents. The formulations may be presented in unit-dose or multi-dose containers, for example sealed ampoules and vials. Extemporaneous injection solutions and suspensions may be prepared from sterile powders, granules and tablets of the kind previously described.

In a preferred embodiment, the drug product of the present invention is in the form of an injectable solution containing a predetermined amount (e.g., 25 mg) of the composition of matter in a sterile lyophilized cake which also contains lactose, sodium citrate, and simethicone. The pH of a solution containing the above ingredients may be adjusted using a suitable buffer (e.g., sodium hydroxide or hydrochloric acid). Reconstitution may be carried out according to known methods, e.g., using a sterile diluent (5 mL) containing 2 percent by volume benzyl alcohol in sterile water. A preferred injectable solution is similar to Premarin® Intravenous made commercially available by Wyeth-Ayerst Laboratories.

The composition of matter also may be formulated such that it is suitable for topical administration (e.g., vaginal cream). These formulations may contain various excipients known to those skilled in the art. Suitable excipients may include, but are not limited to, cetyl esters wax, cetyl alcohol, white wax, glyceryl monostearate, propylene glycol, monostearate, methyl stearate, benzyl alcohol, sodium lauryl sulfate, glycerin, mineral oil, water, carbomer, ethyl alcohol, acrylate adhesives, polyisobutylene adhesives, and silicone adhesives.

In a preferred embodiment, the drug product is in the form of a vaginal cream containing the composition of matter as set forth herein present in a nonliquefying base. The nonliquefying base may contain various inactive ingredients such as, for example, cetyl esters wax, cetyl alcohol, white wax, glyceryl monostearate, propylene glycol, monostearate, methyl stearate, benzyl alcohol, sodium lauryl sulfate, glycerin, and mineral oil. Such composition may be formulated similar to Premarin® Vaginal Cream made commercially available by Wyeth-Ayerst Laboratories.

Dosage units for vaginal or rectal administration may be prepared in the form of suppositories which may contain the composition of matter in a mixture with a neutral fat base polyethylene glycol, or they may be prepared in the form of gelatin-rectal capsules which contain the active substance in a mixture with a vegetable oil or paraffin oil.

The present invention deals with women's health issues. Specifically, the present invention discloses various methods that may be used to treat women's health issues. These women's health issues include the terms “female sexual dysfunction”, “frailty”, “weakness” and “wellness”. More specifically, these terms as used herein include age-related decline in muscle mass and strength and weight. They also include loss of sense of balance, loss of bone strength and loss of resistance to disease. By improving frailty via hormonal replacement therapy a subject is able to have greater energy, better quality of sleep, greater ability to maintain balance and increased feelings of well-being. The present invention may also assist in treating Turner's Syndrome, wasting and osteoporosis.

The androgenic agent of the methods provided preferably is selected to provide the desired effect or effects without causing reactions undesirable in the treatment of female patients. For example, the androgenic agents utilized typically will not cause substantial masculinization, e.g., male pattern baldness, deepening of the voice, changes in libido and a decrease in breast size, among other adverse reactions. Traditional therapy with androgenic agents such as testosterone or methyltestosterone results in masculinization when administered to female patients. These androgens do not have greater anabolic activity than androgenic activity. Thus, the administration of such compounds may cause masculinization in female patients exhibited as, for example, clitoral enlargement, hirsutism, male pattern baldness, deepening of the voice, changes in libido or a decrease in breast size, among others. Such effects must be substantially avoided to provide a satisfactory agent for administration to female patients. The anabolic effects of an androgenic compound as measured by trophic effects on muscles or the reduction of nitrogen excretion may be dissociated from the other androgenic effects.

The methods of the invention may be prescribed for female patients who are in need of treatment or prevention of conditions arising from declining hormone circulation in the body. Typically, the patient will suffer from a number of different symptoms at the same time. Certain conditions or disorders arising from declining hormone production may be effectively treated or prevented by a combination of an estrogenic agent and an androgenic agent. Combinations of these agents may be used to treat or prevent female sexual dysfunction, geriatric wasting or frailty. For elderly women with a variety of symptoms resulting in a poor physical or psychological condition, the present invention includes methods for improving wellness.

The non-aromatizing androgenic agent and estrogenic agent used in the present invention may be administered separately or in a combination formulation, as desired for effective treatment or prevention of the condition or disorder to be treated or prevented. The administration of an androgenic agent and an estrogenic agent according to the methods herein can be sustained for a short duration or may be continued for long term therapy.

Additionally a progestin agent may be used in combination with the invention. Examples of progestin agents are set forth in U.S. Pat. No. Re. 36,247 to Plunkett et al. Examples include, but are not limited to, dl-norgestrel, norethindrone (norethisterone), norethindrone (norethisterone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, cyproterone acetate, desogestrel, levonorgestrel, hydroxyprogesterone caproate, 19-nortestosterone, chlormadinone acetate, megestrol acetate, norgestimate, norgestrel, trimegestone, gestodene, normegestrel acetate, progesterone, 5-pregnan-3, 20-diol sulfate, 5-pregnan-3-ol-20-one, 16,5-pregnen-3-ol-20-one and 4-pregnen-20-ol-3-one-20-sulfate.

The present invention provides a novel therapeutic method and may provide for treatment of menopausal or post-menopausal disorders in a women comprising by either: continuously and uninterruptedly administering an estrogen and a non-aromatizing androgen to a woman, or by continuously and uninterruptedly cyclically administering an estrogen a non-aromatizing androgen to a woman, or by continuously and uninterruptedly on demand administering an estrogen a non-aromatizing androgen to a woman by repetitively using a dosage regimen. Note, however, that the frequency of administration may be greater than once daily, e.g. twice or even three times daily so long as the dosage level as specified herein is not exceeded. Additionally the therapeutic method may comprise a “dosing cycle” wherein the compounds are administered every other day, every third day or once a week. The term “uninterrupted” means that there is no break in the treatment. It should be noted that “cyclical” administration means that there is a break in administration and that, therefore, by definition, cyclical administration cannot be “uninterrupted”.

The present invention is explained in greater detail in the Examples which follow. These examples are intended as illustrative of the invention and are not to be taken are limiting thereof.

### EXAMPLES

### Materials and Methods

Applicants performed a set of experiments to identify the potential for a compound to mimic endogenous sex hormones. An in vivo study was performed using a short-term screening assay to identify changes in weights of male accessory sex glands and tissues in sexually immature castrated male rats. The study preformed is known in the art as an “Oral Hershberger Assay”.

This study included forty-two sexually immature castrated male rats that were randomly assigned to seven different groups and assigned formulations of the test article, oxandrolone, positive control article, 17α-methyl testosterone, or the vehicle, aqueous 0.5% methylcellulose. These formulations were administered orally via gavage once daily for ten consecutive days at dosages of 0 (vehicle), 1, 3 and 10 mg/kg/day. The dosage volume was 0.5 mL/kg, adjusted daily for body weight changes and were given at approximately the same time each day. The subject rats were observed for viability at least twice each day and examined for clinical observations and general appearance weekly during the acclimation period.

Approximately 24 hours after the last dosage administration the rats were euthanized via carbon dioxide asphyxiation. The following tissues were excised trimmed and weighed: liver, paired adrenal glands, paired kidneys, ventral prostate, seminal vesicles together with the coagulating gland, levator ani and bulbocavemous muscles, glands penis and cowper's gland. These tissues were retained in neutral buffered 10% formalin to be used for any possible future evaluation.

The following table presents the percent changes from the control group value.

TABLE 1

| Test Article | Oxandro | Oxandro | Oxandro | Meth-Test | Meth-Test | Meth-Test |
|---|---|---|---|---|---|---|
| Mg/kg/day | 1 | 3 | 10 | 1 | 3 | 10 |
| Seminal Vesicles | 100.7 | 120.1 | 171.0 | 89.6 | 118.0 | 194.4 |
| with Fluid | | | | | | |
| Prostate | 135.3 | 139.4 | 166.3 | 97.0 | 109.1 | 222.5 |
| Prostate (Fixed) | 127.6 | 149.9 | 192.2 | 101.9 | 119.0 | 234.0 |
| Glands Penis | 104.1 | 107.4 | 109.6 | 127.9 | 102.6 | 122.9 |
| Levator Ani and | 107.3 | 148.2 | 192.4 | 111.0 | 114.2 | 139.7 |
| Bulbocavernous | | | | | | |
| Cowpers Gland | 137.1 | 159.7 | 206.5 | 88.7 | 179.0 | 237.1 |

As is evidenced in Table 1, in the majority of the tests, both test articles increased the organ weights of the male accessory sex glands compared to the control group values. The percentage of weight increase in the organs from the oxandrolone dosed rats, were generally equal to, or greater than, the organ weight increases in the 17α-methyl testosterone dosed rats. The methyl testosterone tested rats exhibited a greater increase in weight than that of the oxandrolone dosed rats. This illustrates that the methyl testosterone has a greater effect than oxandrolone. Yet, this study also illustrates the potential of oxandrolone being able to mimic endogenous sex hormones without the typical androgenizing side effects.

These examples are suitable for mice and other animals that have difficultly in swallowing tablets. For use in humans, the preferred embodiments will be tablets, capsules, transdermal patches and the like.

In the specification, there has been disclosed typical preferred embodiments of the invention and, although specific terms are employed, they are used in a generic and descriptive sense only and not for purposes of limitation of the scope of the invention being set forth in the following claims.

What is claimed is:

1. A method of treating sexual dysfunction and/or frailty in a woman comprising:
administering a therapeutic amount of a non-aromatizing androgenic compound in a daily dose in an amount of 0.1 to 10 mg of said non-aromatizing androgenic compound equivalent to oral dosages of oxandrolone of about 0.1 to 10 mg.

2. The method according to claim 1, wherein the non-aromatizing androgenic compound is selected from the group consisting of oxandrolone, oxymetholone, stanozolol, stanozolone, danazol and combinations of any of the foregoing.

3. The method according to claim 1, further comprising the administration of a therapeutic amount of an estrogenic compound.

4. The method according to claim 3, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17α-dihydroequilenin, and mixtures, conjugates and salts thereof.

5. The method according to claim 1, further comprising administering a progestin in a daily dose.

6. A method of treating frailty in a woman undergoing estrogen replacement therapy, the method comprising administering to said woman, continuously and uninterruptedly, a therapeutically effective amount of both estrogen and a non-aromatizing androgen in daily dosages, wherein said estrogenic compound is equivalent to oral estradiol dosages of about 0.05 to 3 mg, and said non-aromatizing androgenic compound is equivalent to oral dosages of about 0.1 to 10 mg of an androgenic compound.

7. The method of claim 6, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

8. The method according to claim 6, wherein the non-aromatizing androgenic compound is selected from the group consisting of oxandrolone, oxymetholone, stanozolol, stanozolone danazol and combinations of any of the foregoing.

9. The method according to claim 6, further comprising administering a progestin in a daily dose.

10. A method of treating frailty in a woman undergoing estrogen replacement therapy comprising cyclically administering to said woman an estrogenic compound, and continuously and uninterruptedly administering to said woman a non-aromatizing androgenic compound.

11. The method according to claim 10, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

12. The method according to claim 10, wherein the non-aromatizing androgenic compound is selected from the group consisting of oxandrolone, oxymetholone, stanozolol, danazol, pharmaceutically acceptable esters and salts thereof, and combinations of any of the foregoing.

13. The method according to claim 10, further comprising administering a progestin in a daily dose.

14. A method of treating weakness in a woman undergoing estrogen replacement therapy comprising continuously and uninterruptedly administering daily dosages of a therapeutically effective amount of an estrogenic compound equivalent to estradiol dosages of about 0.05 to 3 mg, and continuously and uninterruptedly administering to said woman a therapeutically effective amount of a non-aromatizing androgenic compound equivalent to oral dosages of oxandrolone of about 0.1 to 10 mg.

15. The method according to claim 14, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

16. The method according to claim 14, wherein the non-aromatizing androgenic compound is selected from the group consisting of oxandrolone, oxymetholone, stanozolol, danazol and combinations of any of the foregoing.

17. The method according to claim 14, further comprising administering a progestin in a daily dose.

18. A method of treating frailty in a woman undergoing estrogen replacement therapy comprising cyclically administering to said woman a non-aromatizing androgenic compound.

19. The method according to claim 18 further comprising continuously and uninterruptedly administering to said woman an estrogenic compound.

20. The method according to claim 18, wherein the non-aromatizing androgenic compound is selected from the group consisting of oxandrolone, oxymetholone, stanozolol, danazol and combinations of any of the foregoing.

21. The method according to claim 20, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

22. The method according to claim 18, further comprising administering a progestin in a daily dose.

23. A method of treating frailty in a woman undergoing estrogen replacement therapy comprising continuously and uninterruptedly administering daily dosages of a therapeutically effective amount of an estrogenic compound equivalent to estradiol dosages of about 0.05 to 3 mg, and continuously and uninterruptedly cyclically administering to said woman a therapeutically effective amount of a non-aromatizing androgenic compound equivalent to oral oxandrolone dosages of about 0.1 to 10 mg.

24. The method according to claim 23, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17β-dihydroequilenin, and mixtures, conjugates and salts thereof.

25. The method according to claim 23, wherein the non-aromatizing androgenic compound is selected from the group consisting of oxandrolone, oxymetholone, stanozolol, danazol and combinations of any of the foregoing.

26. The method according to claim 23, further comprising administering a progestin in a daily dose.

27. The method according to claim 23, wherein said frailty is selected from the group of muscle tone, balance and bone strength.

28. A method of treating sexual dysfunction and/or frailty in a woman comprising:
administering a therapeutic amount of an androgenic compound in a daily dose in an amount of 0.1 to 10 mg of said androgenic compound, wherein said androgenic compound comprises at least 50% of a non-aromatizing androgenic compound.

29. The method according to claim 28, wherein the non-aromatizing androgenic compound is selected from the group consisting of oxandrolone, oxymetholone, stanozolol, stanozolone, danazol and combinations of any of the foregoing.

30. The method according to claim 28, further comprising the administration of a therapeutic amount of an estrogenic compound.

31. The method according to claim 30, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17α-dihydroequilenin, and mixtures, conjugates and salts thereof.

32. The method according to claim 28, further comprising administering a progestin in a daily dose.

33. A method of treating sexual dysfunction and/or frailty in a woman comprising:
administering a therapeutic amount of a non-aromatizing androgenic compound in a dosing cycle in an amount of 0.1 to 10 mg of said non-aromatizing androgenic compound equivalent to oral dosages of oxandrolone of about 0.1 to 10 mg.

34. The method according to claim 33, wherein the non-aromatizing androgenic compound is selected from the group consisting of oxandrolone, oxymetholone, stanozolol, stanozolone, danazol and combinations of any of the foregoing.

35. The method according to claim 33, further comprising the administration of a therapeutic amount of an estrogenic compound.

36. The method according to claim 33, wherein the estrogenic compound is selected from the group consisting of estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17αΔ^{8,9}-dehydroestradiol, 17βΔ^{8,9}-dehydroestradiol, 6-OH equilenin, 6-OH 17α-dihydroequilenin, ethinyl estradiol, estradiol valerate, 6-OH 17α-dihydroequilenin, and mixtures, conjugates and salts thereof.

37. The method according to claim 33, further comprising administering a progestin in dosing cycle.

38. The method according to claim 33, wherein the non-aromatizing compound is administered daily.

39. The method according to claim 33, wherein the non-aromatizing compound is administered every other day.

40. The method according to claim 33, wherein the non-aromatizing compound is administered every third day.

41. The method according to claim 33, wherein the non-aromatizing compound is administered once a week.
